(19) United States
(12) Patent Application Publication
(10) Pub. No.: US 2003/0139336 A1
(43) Pub. Date: Jul. 24, 2003

(54) Interface patch clamping

(76) Inventors: James Henry Norwood, Cambridge (GB); David Geraint Owen, Cambridge (GB); Voi Piotrowski, Cambridge (GB)

Correspondence Address: FINNEGAN, HENDERSON, FARABOW, GARRETT & DUNNER, LLP, 1300 I STREET, NW, WASHINGTON, DC, 20005, US

(21) Appl. No.: 10/239,046

(22) PCT Filed: Mar. 21, 2001
(86) PCT No.: PCT/GB01/01238

(51) Int. Cl. A61K 38/00

(52) U.S. Cl. 514/12

(57) ABSTRACT

A novel development of the conventional patch clamp technique for measurement of whole cell electrical activity provides for cells to be suspended in a liquid medium at a liquid/air interface (by virtue of the effect of surface tension at the interface) whereby cells are accessible at the interface to a microstructure electrode (such as a pipette tip (3)) to which a cell can attach to form an electrical seal, for the purpose of whole cell voltage clamp recording. The electrode forms a high resistance electrical seal with a cell suspended at the interface without the need to press the cell against a solid support surface. The step of bringing the electrode into contact with the interface is achieved not by relative movement, but by applying a differential pressure across the interface to cause the meniscus to be lowered and so to “bulge” (16) towards and into contact with the electrode. Also provided are apparatus for carrying out the interface patch clamp technique and control logic for operating a computer to carry out the technique.

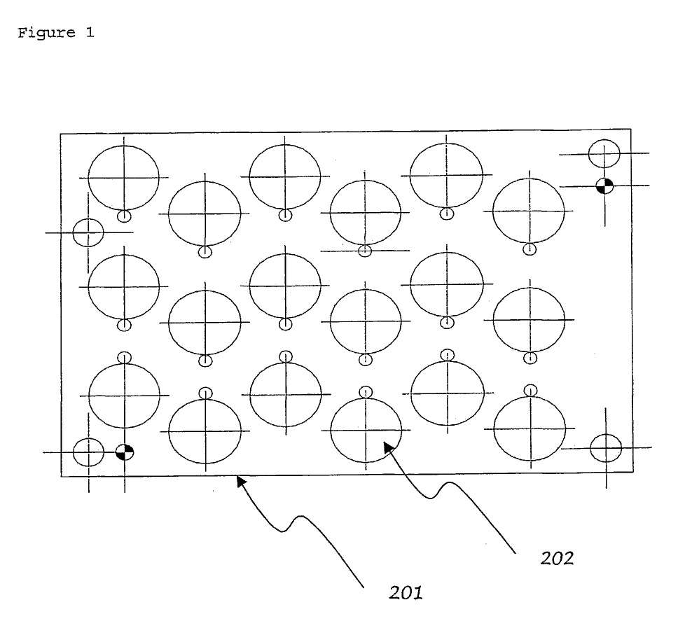

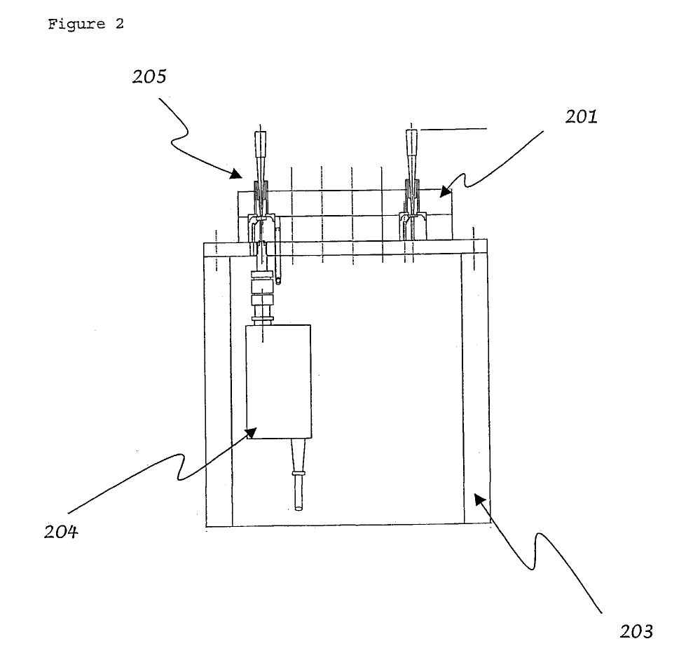

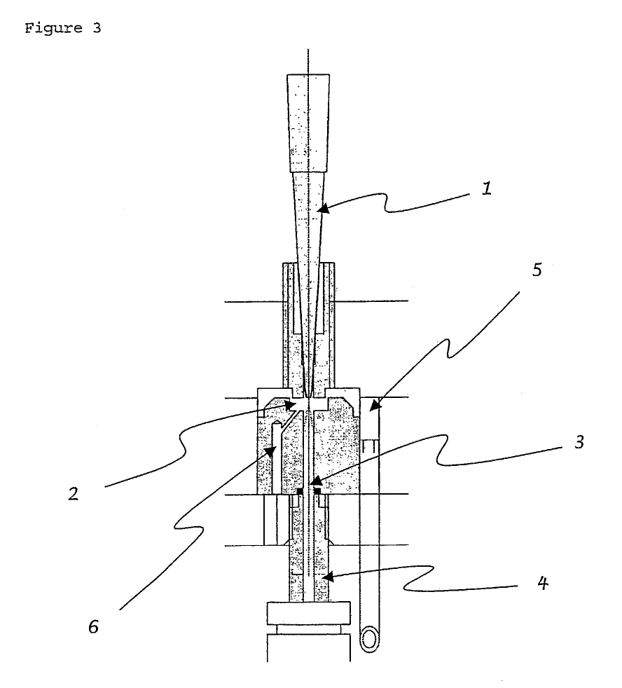

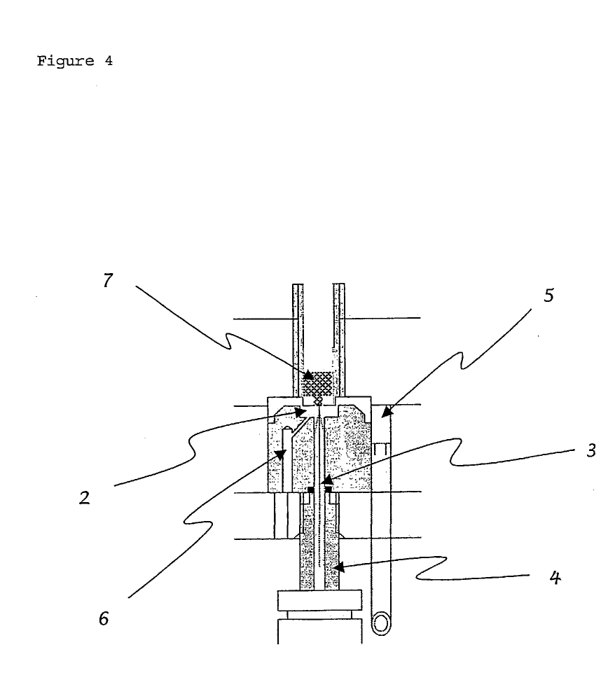

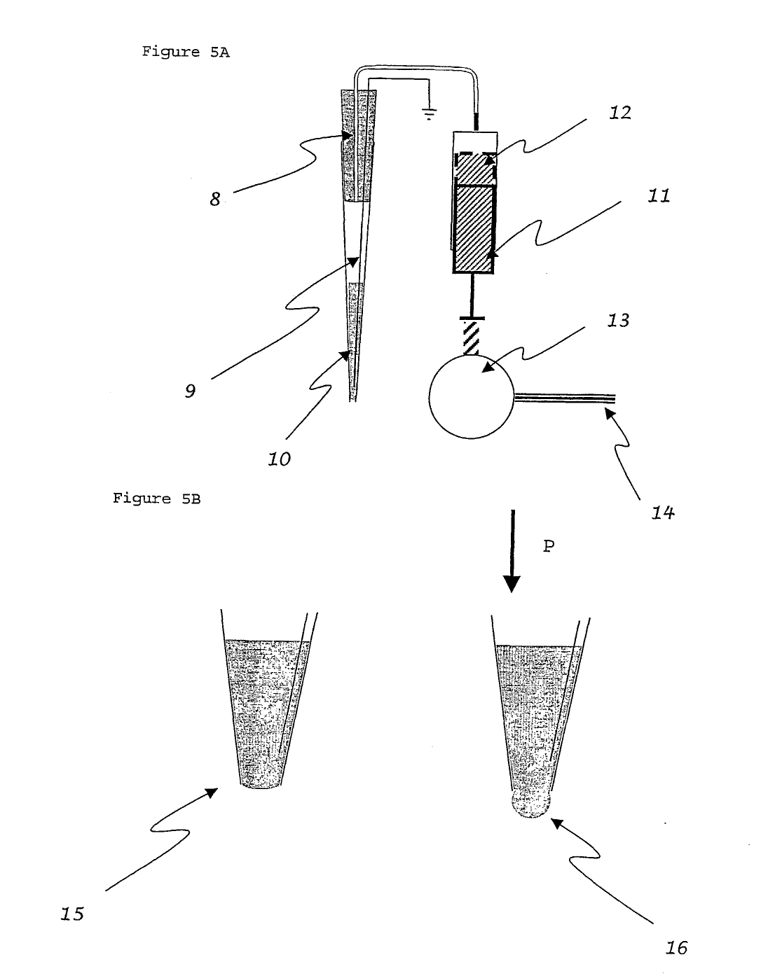

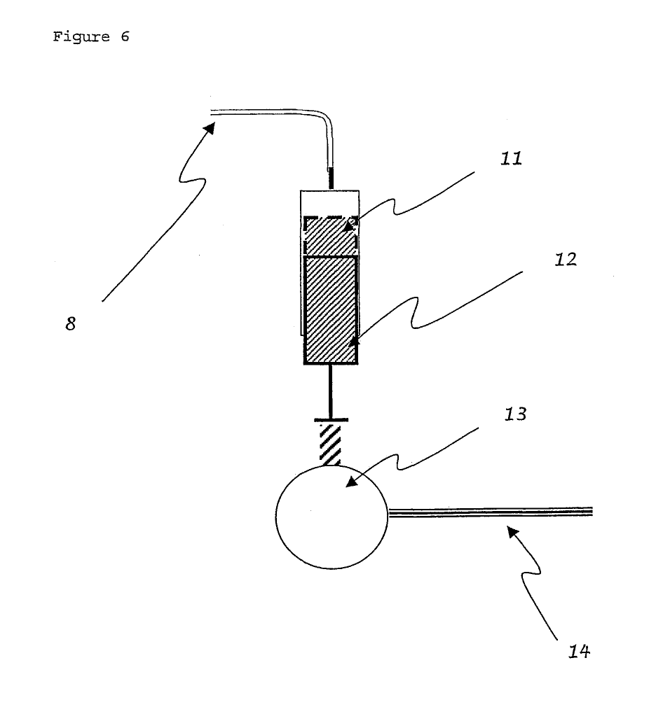

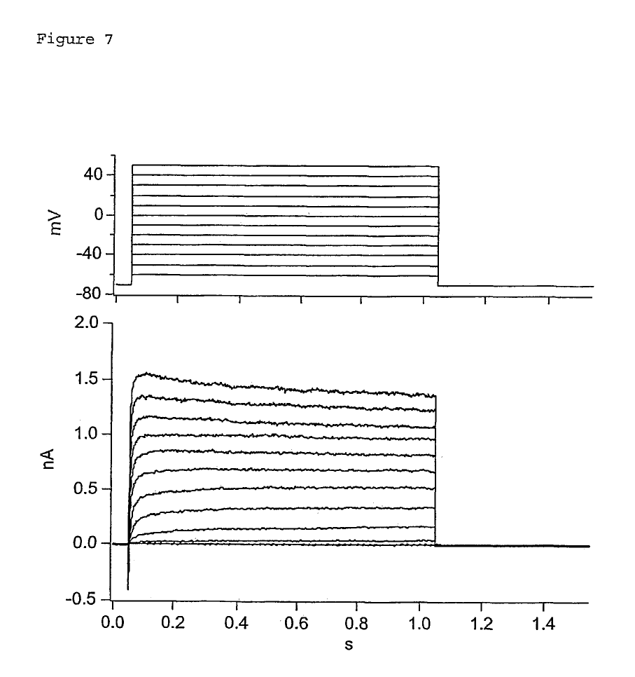

### INTRODUCTION

The present invention provides a novel development of the conventional patch clamp technique. This technique is referred to as the interface patch clamp method.

Voltage gated ion channels are potential targets for a considerable range of novel treatments in a variety of disease states. The development of the patch clamp technique has provided a powerful method for the study of ion channel function and pharmacology in whole cells. However, while the patch clamp technique provides a definitive method for the investigation and screening of drugs with potential activity on voltage gated ion channels, the technique is currently highly dependent on the skill of the operator and tends to be very slow for drug screening. The present invention provides a method for increasing the rate at which compounds may be screened for ion channel blocking/agonist activity using the patch clamp technique. The method can retain the essential features of the conventional patch clamp recording system while facilitating automation of the major time-consuming components of the technique.

### BACKGROUND

Conventional Patch Clamp

The success of the patch clamp technique is derived from the ability to form “tight” (i.e. high resistance: Giga Ohm) electrical seals between an area of the cell membrane (the Patch) and the tip of a pipette. The patch clamp pipette is usually made from glass. The formation of the G-seal is dependent on the profile of the top of the pipette, and is enhanced by the application of suction to the interior of the pipette. The requirements for the formation of the G-seals are well established and the process is usually monitored electrically by display of the current pulse recorded in response to a small voltage step applied throughout seal formation. After formation of a G-seal, the area of membrane under the pipette may be disrupted to obtain whole cell voltage clamp recording mode.

The sequence of events leading to successful G-seal formation and whole cell recording mode using pre-formed patch pipettes is as follows:

1. Selection of a suitable cell.

2. The patch pipette is positioned approximately 50 microns above the cell.

3. The pipette is lowered until the cell surface is deformed by the pipette tip.

4. Negative pressure is applied to the interior of the pipette until a G-seal is formed between the pipette tip and the cell membrane.

5. Whole cell recording mode is established by the application of further negative pressure which disrupts the cell membrane in the area under the pipette tip.

Steps two and three are slow and require considerable manual dexterity and a high level of operator skill. Visualisation of the cells and the patch pipette requires the use of a high quality microscope and, in order to position the pipette, a high quality three axis micromanipulator with sub-micron resolution in each axis is required.

The Invention of PCT/GB99/04073 (WO 00/34776)

In its broadest terms WO 00/34776 provides for one or more cell or cells to be suspended in a liquid medium at a liquid/air interface (by virtue of the effect of surface tension at the interface) whereby the cell or cells are accessible at the interface to a microstructure electrode (such as a pipette tip) to which a cell can attach to form an electrical seal, for the purpose of whole cell voltage clamp recording. According to the invention the electrode can be caused to form a high resistance electrical seal with a cell suspended in the liquid at the liquid/air interface without the need to press the cell against a solid support surface.

Any body of liquid or column of liquid, which gives rise to a situation in which a cell or cells are located in the liquid at a liquid/air interface, can be used in the invention. For example cells may be suspended in a column of liquid held by surface tension in a capillary tube. Alternatively cells may be suspended in a droplet of liquid, which droplet may itself be suspended from or supported by a support.

It will readily be appreciate that the interface patch clamp technique can be operated in “single cell mode”, or could be multiplexed to operate on a matrix of cells with multiple electrodes.

According to one aspect of the invention, interface patching can utilise a patch pipette of conventional type. Cells are supported on a liquid/air interface at one end of a capillary tube (e.g. made of glass, polyethylene or other suitable material). The axis of the patch pipette is in line with the axis of the tube so that the pipette tip can be manipulated into the opening of the tube where the cells are supported at the air/liquid interface. The capillary tube or the patch pipette can be mounted onto a single axis manipulator. Only one manipulator is required and this may be used to move either the patch pipette or the capillary tube. Whole cell recording mode is established as follows:

6. A layer of cells is established at the interface between the extracellular physiological solution (the liquid in which the cells are suspended) and air by dipping the capillary tube into a suspension of cells. The density of cells in the suspension must be sufficient to provide a sufficient number of cells to form a layer of cells at the interface.

7. Electrical contact with the extracellular solution is established via a non-polarizable electrode (e.g. an Ag/AgCl wire) and the tube is mounted either to a fixed clamp or single axis manipulator.

8. A patch pipette is provided which can be filled with electrolyte solution.

9. The patch pipette is mounted concentrically with the capillary tube either via a single axis manipulator or fixed clamp (if the capillary tube is to be moved). The pipette filling solution is connected via the non-polarizable electrode to the headstage of a conventional patch clamp amplifier. The pipette holder allows suction to be applied to the pipette interior.

10. Cell attached patch mode of recording is established by bringing the pipette tip in contact with the interface by moving the pipette and the capillary tube respectively together along the single mounting axis (e.g. either by moving the pipette towards the tube and interface or vice versa). On entry into the interface the movement of the pipette and capillary tube together is stopped and the pipette current is offset to zero on the patch clamp amplifier. The resistance of the pipette increases when the pipette contacts one of the cells at the air/liquid interface. Suction is then applied to the interior of the pipette and the pipette and capillary tube are moved closer together until the pipette tip is located inside the capillary tube.

Initial seal formation between the pipette tip and the cell may also be assisted by the application of gentle suction during entry of the pipette into the interface.

A G-seal is formed between the patch pipette tip and the cell membrane by the application of further suction to the interior of the pipette and monitoring the pipette resistance.

11. Following the formation of cell attached patch mode, the suction is released, pipette current is offset to zero and a holding voltage applied to the pipette (e.g. −60 mV).

12. A whole cell recording is obtained by the application of further suction to the pipette interior until the whole cell recording mode is established in conventional manner.

According to this invention it is preferred that the capillary tube should be mounted in an upright orientation (i.e. essentially vertically) with the air/liquid interface at the downward end of the tube.

This has the advantage that suspended cells will tend to “sediment” naturally to the downward end of the tube and be collected there in a layer. The layer will preferably be several cells deep and loosely packed. Thus according to the invention the pipette tip may be moved upwardly relative to the air/liquid interface at the tube end (either by moving the pipette or the tube along the single axis) so as to come into contact with a cell in the layer at the interface. The relative density or concentration of cells at the interface compared to the density in the bulk of the liquid in the tube ensures a high probability that a cell can be collected on the tip without the need for visualisation of the operation and without the need for multidirectional manipulation of the tip/cell positional relationship. Surprisingly it has been found that G-seal formation between the cell and the pipette can occur without pressing the cell against a solid substrate.

Where the arrangement is intended to operate with the pipette in an upright orientation (i.e. essentially vertically) with the tip uppermost and pointing upwardly, the pipette should be constructed so as to prevent the filling electrolyte solution flowing out and being lost. This may he achieved for example by use of a custom-made mounting assembly and/or by shaping the pipette body to prevent loss of filling solution (e.g by bending the pipette shaft into a U- or J-shape).

The invention also provides methods and apparatus employing control logic to allow automation of a patch clamp system employing the Interface Patch Clamp technique described herein. The logic described will control one or more electromechanical micromanipulators/translators holding one or more patch clamp pipettes and/or capillary tubes in order to patch clamp cells and apply drugs/compounds in order to screen for activity on membrane ion channels. A major advantage of the logic described is that automation is achieved in this system by the use of feedback from signals from the patch clamp amplifier and no image recognition software is required.

It will be readily appreciated by those skilled in the art from the teaching of WO 00/34776 that:

1. The stability of recording using the interface patch clamp technique may be superior to that of conventional patch clamping. The greater stability of interface patch clamping is because the cell is held by the patch pipette alone. In conventional patch clamp recordings the cell is held by the path pipette and a solid substrate and vibration tends to move the pipette relative to the substrate causing loss of the G-seal. The interface patch clamp is, in contrast to conventional patch clamp apparatus, relatively insensitive to vibration during drug application.

2. This method of drug application could be applied to a plurality of recording pipettes/capillaries and form the basis for a high throughput electrophysiological assay system. It will readily be appreciated that the Interface Patch Clamp technique could be used with multiple pipettes and multiple capillaries in a manner in which each pipette enters its respective aligned capillary either individually in sequence or all together. Although not currently preferred, a single pipette could be used which is caused to enter more than one capillary sequentially. Multiple patch clamp recordings could be made either sequentially or simultaneously, depending on the application.

As was mentioned above, it is not essential to the general principle of the invention to use a capillary in order to create a column of liquid which gives rise to a liquid/air interface at which cells can be located. Other ways can be envisaged in which the same effect can be achieved. For example, a droplet or “blob” of liquid may be provided on a support surface. The surface has a hole through it and the droplet covers the hole. Surface tension prevents the liquid from the droplet dropping through the hol3. Within the droplet cells are suspended. This allows access to the droplet and the cells contained therein by a suitable electrode such as a patch pipette. Means may be provided for flow of other liquids in to and out of a dish or other container of which the support surface with the hole in it forms a wall. Once a cell has been attached to the electrode, other liquids may be introduced into the container either in batch mode or in flow-through mode in order to result in the cell being exposed at its external surface to the surrounding liquid. Clearly in such an arrangement, the original liquid and the remaining un-attached cells will tend to be washed away from the area of the electrode/pipette.

Droplets might be provided on non-perforate support surfaces. The effect of surface tension may be to allow droplets of a suitable liquid to adhere automatically to the underside of a suitable support surface. The support surface might for example be a cover slip of glass or other material. Droplets in which cells are suspended provide the air/liquid interface and consequently may be used in a method of interface pathc clamping as described above.

The arrangement allows for the formation of a matrix of cell suspensions so that multiple electrodes can be multiplexed to take readings either simultaneously or sequentially (as well as singly).

It will be appreciated by those skilled in the art that a conventional glass “patch pipette” could be replaced by an equivalent electrode. The electrode might be either a single region or a matrix of regions on a sheet of material (such as a silicon wafer) which incorporates a microstructure to which a cell can be attached and which would provide the necessary electrical connection. Mictostructures could be etched on to a silicon wafer (e.g. an oxidised silicon wafer), which microstructures would be designed and adapted to be able to capture a cell from the liquid/air interface of an arrangement according to the present invention. Thus, the performance and advantage of the invention is not limited to the currently preferred conventional glass patch pipette but would include functionally equivalent means.

A drug in liquid solution can be applied to the cell in a number of ways. For example the drug could be applied via the capillary if the air interface is formed in a capillary tube. Alternatively the drug can be applied by perfusion into a dish. Furthermore, perfusion could be achieved by flowing the drug-containing liquid through a dish or container.

A further arrangement for drug application is described in WO 00/34776. In this case the electrode (for example the patch pipette) penetrates through the lower wall of a well. A suspension of cells is loaded in to a capillary tube as previously described. Attachment of a single cell to each pipette tip follows, as described before. Once cells are attached to the pipette tips the capillary tubes containing the remainder of the cells in suspension can be removed. Subsequently, a drug solution is dispensed into each well and patch clamp measurements can then be carried out on the cell in the environment of the surrounding drug solution.

Optimisation of Patch Clamping Conditions

Those skilled in the art will appreciate that within the general teaching for the interface patch clamping method and apparatus, it may be necessary to optimise certain conditions for patch clamp measurements. For example the concentration and packing density of cells in the suspension may need to be optimised. Furthermore, the cells and/or solutions may be temperature sensitive and an optimum temperature of operation may need to be determined. Since the technique relies on the formation of a liquid/air interface at which the cells are located, it may be necessary to optimise the osmolarity of the suspending liquid medium in order to achieve the optimum level of surface tension etc.

### SUMMARY OF THE INVENTION

Where legally permissible the content of PCT/GB99/04073 (WO 00/34776) is explicitly incorporated herein by way of references

The present invention can provide an improved method for operating the Interface Patch Clamp technique generally described in PCT/GB99/04073.

According to the present invention the Interface Patch Clamp technique is modified in that the step of bringing the microstructure electrode (pipette tip) into contact with the interface is achieved not by relative movement of the parts of the equipment, but by applying a differential pressure across the liquid/air interface to cause the meniscus to be lowered and so to cause the surface of the liquid/air interface to “bulge” towards and into contact with the electrode.

The term “lower the meniscus” means that the radius of curvature of the surface of the liquid droplet becomes greater, and the droplet expands.

It will be appreciated that one or more mechanical manipulator may be employed, according to this invention, to bring the respective parts of the patch clamp equipment into close proximity, provided that the final relative movement of the electrode and the interface are cause by the applied pressure differential. Alternatively, according to the invention, a part of the equipment holding the electrode may be mated with, attached to or held together with a part of the equipment comprising the interface in any suitable arrangement allowing a small gap between the electrode and interface in the absence of an applied pressure differential.

The term “pressure differential across the interface” means that the hydrostatic pressure in the liquid phase (inside the droplet) differs from the air pressure ambient at the outer surface of the meniscus. In order to cause a lowering of the meniscus the internal liquid phase pressure is raised above the external ambient air pressure. Alternatively the same effect can be achieved by a relative lowering of the ambient air pressure. The meniscus surface level can be caused to move towards and come into contact with the electrode pipette tip by applying a (small) increase in air pressure (by any suitable means) above the liquid in the capillary. The same effect could be achieved by increasing the volume of liquid in the capillary or by driving the liquid down the tube (e.g. by use of a piston or plunger).

It will be apparent that the invention could be performed by a relative reduction in pressure around the capillary tip; for example by mounting the capillary tip region in a sealed housing having a controllable interior housing pressure.

As with the invention of the Interface Patch Clamp technique described in PCT/GB99/04073, the present invention may be employed to make single cell recordings, or may be applied to arrays in which multiple single cells are attached to multiple electrodes.

An advantage of the present invention is that once the equipment has been set up with the interface near to the electrode (e.g. by mechanical or physical manipulation and positioning), the actual step of making contact with the interface (and hence with a cell) by the electrode involves no moving parts and can be sensitively pressure-controlled. This is especially useful for patch clamping in large arrays for High Throughput Screening. Clearly, the technique can employ a means for overall pressure-control and may be designed to allow pressure-control of individual elements of an array.

It will be a matter for optimisation of any particular patch clamp arrangement to set up the equipment so that the meniscus can be lowered enough to come into contact with the electrode, without breaking the surface tension. It is important that the meniscus stays intact during the initial contact with the interface, up to the point at which a giga-ohm-seal is formed with a cell. After that stage has been is successfully reached it is possible (as an additional advantageous feature of the invention) that the relative differential pressure may be maintained or further increased so as to cause the remaining liquid (and remaining cells) in the liquid phase to be ejected from the tube. This may permit an easy way of replacing the first cell-containing liquid with another liquid (e.g. containing an active substance for testing/screening).

The invention is illustrated by way of example with reference to the figures, in which:

FIG. +1

Aerial view of plexiglass multiwell plate (201) showing the position of 1 of the 18 wells (202).

FIG. +2

Side view of recording assembly showing positions of multiwell plate (201), support stand (203), patch-clamp headstage (204) and cell application assembly (205).

FIG. +3

Cross section of cell application system wherein cells are applied via a disposable pipette tip (1) and recording chamber (2) also showing position of recording pipette (3), pipette holder (4), overflow channel (5) and earth wire access port (6).

FIG. +4

Cross section of an alternative cell application system where cells are pipetted directly into chamber (7). Recording chamber (2), recording pipette (3), pipette holder (4), overflow channel (5) and earth wire access port (6).

FIG. +5

A) Cell applicator consisting of pressure line (8), earth wire (9), suspension of cells (10), driven syringe in starting position (11), syringe in active position (12), stepper motor (13), serial communication line to computer (14). B) Illustration of movement of meniscus from (15) to (16) as pressure (P) is increased due to movement of driven syringe from starting position (11) to active position (12).

FIG. +6

Suction control system showing starting position of driven syringe (11), active position of driven syringe (12), stepper motor (13), serial communication line to computer (14) and pressure/vacuum line to patch-pipette holder (8).

FIG. +7

Exemplary recording made using the pressure controlled interface patch-clamp illustrated in FIGS. +1-6. The configuration used for the cell applicator was as illustrated in FIG. +3. Kv1.1 potassium channels were expressed in a Chinese hamster ovary cells and recordings made using standard patch-pipette filled with (in mM): 100 Kgluconate, 20 KCl, 1CaCl₂, 1MgCl₂, 10HEPES, 11EGTA-KOH, 5ATP-Na₂, 2GSH, pH 7.2 and a cell bathing solution consisting of (in mM): 140NaCl, 2.5KCl, 2MgCl₂, 2CaCl₂, 10HEPES, 10glucose, sucrose to 320mOsm, pH 7.4. Top: superimposed series of voltage steps used to activate the Kv1.1 channel. Bottom: superimposed whole-cell Kv1.1 currents recorded in response to voltage steps.

### EXAMPLE

Mode of Operation of Cell Applicator

1. The cell applicator comprises a suitably shaped adaptor containing a length of small-bore tubing and a silver/silver chloride reference electrode fashioned into-an integrated leakproof assembly. The other end of the tubing is connected to a gas tight syringe which can be driven by a computer controlled motor such as a stepper motor (FIG. +5)

2. The tip of the cell applicator is placed into a suspension of cells and a sample of said cells is aspirated by withdrawal of the piston.

3. The cell applicator is placed into position above and concentric with the tip of the patch pipette (FIG. +3). Cell suspension can also be applied directly into a channel in the top plate as in FIG. +4.

4. The suction device is commanded to provide a small amount of suction to the interior of the patch electrode. Concomitantly, the piston in the cell applicator is advanced manually or automatically in such a manner that the interface of the cell-containing saline solution approaches slowly towards the patch pipette (FIG. +5B). Automatic operation of the cell applicator would be analogous to the suction control operation below.

5. When contact between the patch pipette and the interface is established (measured by the passage of current in the nA range on passing a small voltage pulse), movement of the interface is continued for a small distance to ensure that the patch pipette is immersed in the cell suspension.

6. When the resistance measured between pipette and cell suspension falls into the region 200M to 3G the whole cell mode of recording is established by the application of a greater degree of suction to the interior of the patch pipette.

Mode of Operation of Suction Control Device

1. The suction device is computer controlled and utilises an RS-232 serial communications protocol to operate the linear motion stepper motor. Solenoid valve speed and distance of stepper motor movements are established by sending appropriate command strings from the computer to the interface electronics assembly (FIG. +6)

2. On first use, the suction device is initialised by sending commands to obtain the following sequence:

a) open the solenoid valve and to drive the stepper motor until the end of travel of the piston air displacement system is reached.

b) close the solenoid valve and drive the stepper motor to the first (lowest suction) position.

3. The degree of suction may subsequently be varied by sending the appropriate command strings from the computer to the suction control device such that the air displacement piston is moved to varying degrees from the position reached at the end of the initialisation process.

1. A method for providing a cell attached to a patch clamp electrode and having a high resistance (Giga Ohm) electrical seal between an area of the cell membrane and the electrode, which includes the steps of:
i) providing a suspension of cells in a liquid;
ii) causing the formation of a layer of cells at the interface between the air and the liquid in which the cells are suspended;
iii) bringing the patch clamp electrode into contact with the interface by applying a differential pressure across the liquid/air interface;
iv) contacting the electrode with a cell in the cell layer at or near the interface; and
v) causing attachment of the cell to the electrode.

2. A method according to claim 1 for providing a cell attached to the tip of a patch clamp pipette and having a high resistance (Giga Ohm) electrical seal between an area of the cell membrane and the tip, which includes the steps of:
i) providing a capillary tube containing a suspension of cells in a liquid;
ii) causing the formation of a layer of cells at one end of the capillary tube at the interface between the air and the liquid in which the cells are suspended;
iii) bringing the tip of the patch clamp pipette close to contact with the interface by moving one or both of the pipette and the tube respectively together along a common axis of movement;
iv) applying a differential pressure across the liquid/air interface;
v) contacting the tip with a cell in the cell layer at or near the interface; and
vi) causing attachment of the cell to the tip.

3. A method according to claim 1 or 2 in which the liquid in which the cells are suspended is an extracellular physiological solution.

4. A method according to claim 1 or 2 in which the layer of cells is several cells deep and loosely packed.

5. A method according to claim 2 in which the layer of cells is formed by mounting the capillary tube in an essentially upright orientation and allowing the suspended cells to sediment to the downward end of the tube to collect there in a layer.

6. A method according to claim 2 in which the capillary tube is mounted essentially upright with the interface at a lower open end of the tube and the pipette is mounted essentially upright with the tip upwardly pointing.

7. A method according to claim 2 in which the capillary tube and pipette are concentrically mounted with the capillary tube in a fixed position and the pipette movable along the common axis.

8. A method according to claim 2 in which the capillary tube and pipette are concentrically mounted with the pipette in a fixed position and the capillary tube movable along the common axis.

9. A method according to claim 2 wherein gentle suction is applied to the pipette during contact with the interface and during the step of contacting the tip with a cell.

10. A method according to any preceding claim, in which contact between the pipette tip and the air/liquid interface and/or subsequent movement of the pipette tip into the liquid is detected by monitoring pipette capacitance.

11. A method according to any preceding claim, in which if no cell is contacted at or near the interface as or within a predetermined time after contact between the pipette and the interface, the pipette is withdrawn from the interface and then moved back to the interface to repeat the attempt to contact a cell.

12. An apparatus for carrying out the method of any preceding claim which is a computer controlled apparatus including the following elements:
i) a patch clamp amplifier;
ii) a source of variable suction for a patch clamp pipette under the control of the patch clamp amplifier;
iii) a holder for a capillary tube to be mounted vertically;
iv) a holder for a patch clamp pipette to be mounted vertically in the same axis as the capillary tube in an inverted orientation with the tip pointing upwardly;
v) a manipulator for controlling relative movement of the capillary tube and pipette along a common axis of movement under feedback control from the patch clamp amplifier and allowing for the tip of the pipette to enter a downwardly facing end of the capillary tube;
vi) means for applying a differential pressure across the liquid/air interface;

13. An apparatus according to claim 12 which includes an array of a multiplicity of capillary tubes and an array of a multiplicity of pipettes.

14. An apparatus according to claim 12 or 13, comprising a pipette capacitance sensor for sensing pipette capacitance as the pipette tip contacts an air/liquid interface at the end of the capillary tube and enters the liquid in the capillary tube during operation of the apparatus.

15. A computer-program-controlled patch clamping process for carrying out the method of any of claims 1 to 11.

16. A computer-program-controlled patch clamping process for controlling the apparatus of claim 12, 13 or 14.

17. A computer-readable medium carrying a computer program for controlling a computer to implement the method any of claims 1 to 11 or to control the apparatus of claim 12, 13 or 14.

18. A method for controlling a computer by means of a computer program for implementing the method of any of claims 1 to 11.
